(19) United States
(12) Patent Application Publication
(10) Pub. No.: US 2023/0103675 A1
(43) Pub. Date: Apr. 6, 2023

(54) ALLERGEN PROTECTION FOR THE SKIN

(71) Applicant: BEIERSDORF AG, Hamburg (DE)

(72) Inventors: Joern Hendrik REUTER, Henstedt Ulzburg (DE); Wilfried SIEFKEN, Hamburg (DE); Judith SEIDEL, Berlin (DE); Anna RINAS, Hamburg (DE); Yvonne WOEHRMANN, Norderstedt (DE); Marlis RIEDEL, Hamburg (DE); Mirko TESCH, Hamburg (DE); Christoph ERNST, Hamburg (DE); Tanja BUSSMANN, Hamburg (DE)

(21) Appl. No.: 17/759,634

(22) PCT Filed: Jan. 26, 2021
(86) PCT No.: PCT/EP2021/051736

§ 371 Date: Jul. 28, 2022

(30) Foreign Application Priority Data
Jan. 29, 2020 (DE) 10 2020 201 019.6

(51) Int. Cl. A61K 8/64; A61Q 17/00

(52) U.S. Cl. CPC A61K 8/64; A61Q 17/00

(57) ABSTRACT

Allergenic substances, allergens, are intercepted by peptides that consist of 2-10 amino acids, at least one amino acid having one or more reactive side chains. This barrier effect of the peptides, in particular in topically administrable preparations, allows for a new antiallergic form of therapy.

The invention is a preparation comprising specific peptides that can bind allergens, and the use of peptides to prevent or reduce the penetration or accumulation of allergens in or on the skin.

The skin forms a barrier that protects the human body from liquid or solid foreign substances. Nevertheless, foreign substances and thus potential allergens are still able to penetrate. This can trigger an immune response, causing the affected area of skin to become inflamed.

Normally, the immune system, which is made up of antibodies, white blood cells, mast cells, complement proteins, and other substances, defends the body against foreign substances (also termed antigens). On contact with certain substances (allergens) in the environment, food, or medicaments that are harmless to most people, the immune system of susceptible people can overreact. The result is an allergic reaction. Some people are allergic only to a single substance. Others in turn have an allergic reaction to many substances. About one quarter of all German citizens suffer from allergies.

Allergens can trigger an allergic reaction when coming into contact with the skin or eyes (or if they are inhaled, eaten, or injected). An allergic reaction can occur in different forms.

Penetrating substances, such as small chemical molecules, pollen, allergens, metal ions, but also proteases, can trigger pathologies such as atopic dermatitis or allergic contact dermatitis, which are triggered by reactions with molecular constituents of the skin or of the immune system.

At the present time, it is only the symptoms triggered by the penetration of foreign substances that are treated.

Allergens are substances that the body recognizes as foreign and that trigger an excessive immune response, an allergy. There are plant allergens such as pollen, animal allergens such as cat hair, and chemical allergens such as preservatives.

An allergen is a substance that can trigger hypersensitivity (allergic) reactions through the mediation of the immune system. An allergen is an antigen. Allergens have no chemical commonalities. It is therefore difficult to develop a chemical that is able to combat or destroy all allergens at the same time.

Most allergens are proteins or protein compounds. On contact with allergens, the immune system of allergic patients responds by forming IgE antibodies. “Pseudoallergens”, on the other hand, are substances in which the immune system is not involved, but which are mediators, for example histamines.

Medicine distinguishes between four types of allergies, depending on which immunological responses the allergen causes. The first three types of allergies are mediated by body fluids and by antibodies and other factors (humoral) contained therein; the fourth type of allergy proceeds on the other hand via a cell-mediated pathway (via T cells). There are however also mixed pictures.
Type I: Allergy of immediate type
Type II: Cytotoxic reaction
Type III: Immune complex formation
Type IV: Cellular immune response

Type I includes for example allergic rhinitis, nettle rash (urticaria) or asthma symptoms, but also pollen allergies, insect venom allergies, and some drug or food allergies. Immediate-type allergic reactions often have a severe course. If a reaction to an allergen is particularly strong, the massive release of histamine can also trigger anaphylactic shock.

In type I allergy, the body forms corresponding group IgE (immunoglobulin E) antibodies immediately after contact with a specific allergen, the immune system having now been sensitized toward these antigens. The next time there is contact with an allergen, the IgE antibodies form a linkage to the so-called mast cells that are found in the skin and mucous membranes. They then bind the antigens to themselves. This bridging (between mast cell, antibody, and antigen) causes the mast cells to release the inflammatory mediator histamine (mast cell degranulation). The histamine and other substances released cause the uncomfortable symptoms typical of allergies. In the immediate vicinity, the histamine released causes blood vessels to dilate with the result that they become more permeable. The inflow of blood intensifies in order to be able to transport away foreign matter and waste. This leads to reddening and swelling of the skin or mucous membrane. Itching is also a frequent consequence of the inflammatory reaction.

Type IV, also termed delayed type, mainly triggers allergy symptoms on the skin, as is the case for example in nickel allergy. Contact allergies are generally responsible for a large proportion of cases resulting in incapacity for work.

Allergies of this type can develop unnoticed over years, but then suddenly trigger very violent immune reactions. In this case, foreign bodies that have penetrated are attacked directly by T-lymphocytes together with helper cells.

Contact allergens are triggers of type IV allergies. The typical clinical picture is allergic contact dermatitis, which appears on the exact same parts of the body that come into contact with the allergen concerned. Usually these are the hands, the face, the lower legs or the neck.

The most common contact allergens include nickel, thiomersal, perfume, cobalt, formaldehyde, balsam of Peru, rosin, isothiazolinones, chromium or thiuram mix.

Also known is the group of plant contact allergens, the urushiols, which represent a major problem particularly in the geographic range, for example in North America, of poison ivy, poison oak, and sumac, as representatives of the Anacardiaceae family.

Since there is no universal remedy against allergens, it would be desirable to prevent or at least reduce the penetration or accumulation of substances on or in the skin or the body.

Intercepting the active allergens and thus preventing the immune response would therefore be a preferred treatment.

The term peptide describes a molecule that is constructed from amino acids and that are linked to one another via peptide bonds. Peptides are systematically classified according to the number of amino acids from which they are constructed.

Thus, oligopeptides contain fewer than 10 amino acids, for example di- or tripeptides.

Polypeptides, on the other hand, contain a larger number of amino acids. Long polypeptide chains are also referred to as proteins.

In the condensation of amino acids, the carboxy group of one amino acid formally reacts with the amino group of the other amino acid to form the acid amide moiety —CO—NH—, with elimination of water. The newly formed amide bond between the carbon atom of the carbonyl group and the nitrogen atom thus becomes a peptide bond.

The 20 canonical amino acids are listed in the table below.

Amino acid

| Name | Abbrev. | Symbol | Acyl group |
|---|---|---|---|
| Alanine | Ala | A | Alanyl- |
| Arginine | Arg | R | Arginyl- |
| Asparagine | Asn | N | Asparaginyl- |
| Aspartic acid | Asp | D | α-Aspartyl- |
| Cysteine | Cys | C | Cysteinyl- |
| Glutamine | Gln | Q | Glutaminyl- |
| Glutamic acid | Glu | E | α-Glutamyl- |
| Glycine | Gly | G | Glycyl- |
| Histidine | His | H | Histidyl- |
| Isoleucine | Ile | I | Isoleucyl- |
| Leucine | Leu | L | Leucyl- |
| Lysine | Lys | K | Lysyl- |
| Methionine | Met | M | Methionyl- |
| Phenylalanine | Phe | F | Phenylalanyl- |
| Proline | Pro | P | Prolyl- |
| Serine | Ser | S | Seryl- |
| Threonine | Thr | T | Threonyl- |
| Tryptophan | Trp | W | Tryptophyl- |
| Tyrosine | Tyr | Y | Tyrosyl- |
| Valine | Val | V | Valyl- |

In addition to the 20 proteinogenic amino acids, there are a whole series of other amino acids in nature that are referred to as non-proteinogenic amino acids.

There are also a number of amino acids that are chemically modified only after they have been incorporated into the growing polypeptide chain. These modified amino acids thus arise from proteinogenic amino acids by post-translational modification. One exception in some organisms is selenocysteine, in which the S atom in cysteine is replaced by selenium. Selenocysteine has its own tRNA and a codon that is otherwise regarded as a stop codon in the genetic code, which means that selenocysteine can in fact be counted as one of the proteinogenic amino acids. This amino acid occurs for example in the formate dehydrogenase of the bacterium E. coli and in mammalian glutathione peroxidase.

Homocysteine, L-homocysteine (Hcy), is also a naturally occurring but non-proteinogenic α-amino acid.

There are in nature 20 standard amino acids that, aside from a few exceptions, make up every protein. This accordingly places limits on the biochemical, biophysical, and reactive properties of desired functional proteins and peptides. However, there are now methods for introducing chemically modified amino acids having a wide variety of reactive groups in site-specific positions. A basis therefor achieved is for example the methodology of amber suppression or a coupling of reactive groups via click chemistry.

In cosmetics, peptides are used inter alia to improve skin characteristics. They are commonly used to reduce the depth of wrinkles. There are however also other possible uses, for example peptides that have a beneficial effect on the water content of the skin by improving the synthesis of the water channels in the skin.

WO 2012164488 A2 describes a mixture of a tripeptide and a tetrapeptide for therapeutic treatment of the papillary dermis and to prevent and/or treat skin aging, especially photo-induced aging.

EP 979829 A2 describes for example the use of oligopeptides for cosmetic and topical dermatological skin lightening or for preventing skin tanning.

Peptides are known to be susceptible to oxidation reactions and/or degradation reactions, which are made noticeable through discolorations or off-odors, which makes their incorporation and use in cosmetic preparations problematic.

Increasingly adverse environmental influences and a greater abundance of foreign substances are coming into contact with human skin and can lead to discomfort, skin irritation, and pathologies.

Besides allergies, foreign substances such as allergens can also cause dermatologically relevant skin changes, chronic conditions such as atopic dermatitis.

It is therefore additionally desirable to provide a barrier against such foreign substances, in particular an allergen barrier.

It is also desirable that the barrier system is easy to apply, permits a broad range of applications, and is designed to be stable, easy to store and easy to use.

One embodiment of the invention comprises preparations for topical application comprising one or more peptides for preventing or attenuating allergic reactions in or on the skin. The allergic reactions are here caused by the penetration and/or accumulation of foreign substances and/or allergens on and/or in the skin. The peptides of the invention are characterized in that they contain 2 to 10 amino acids and that at least one of these amino acids has one or more reactive side chains.

A further embodiment of the invention is a preparation for topical application comprising one or more peptides for preventing or reducing the penetration and/or accumulation of foreign substances and/or allergens on and/or in the skin.

The peptides form a skin barrier for foreign substances. The peptides thus result in particular in the prevention or attenuation of allergic reactions in or on the skin. Preference is thus also given in accordance with the invention to the prevention or attenuation of atopic dermatitis and/or allergic contact dermatitis by the peptides of the invention or by the preparations in which they are present.

One embodiment of the invention therefore comprises also peptides for use in the treatment of atopic dermatitis and/or allergic contact dermatitis.

The peptides of the invention can be used as scavengers against foreign substances.

The peptides of the invention advantageously bind contact allergens. A contact allergy is an allergy to mostly small-molecular-weight substances known as contact allergens. These commonly occur in everyday life or at the workplace. It is based on a T-cell-mediated specific response of the immune system (type IV/delayed-type sensitization) to reactive substances that, as so-called haptens, generally become an allergen only after binding to proteins in the skin. Known contact allergens include inter alia metals (for example nickel), preservatives, fragrances and dyes, rubber constituents, constituents of resins, adhesives, monomers of plastics (for example epoxy resin, acrylates), disinfectants, and particular natural substances (for example plant substances). Contact allergy is manifested clinically as allergic contact dermatitis. Contact dermatitis (also termed contact eczema) is an inflammatory, non-infectious intolerance reaction of the skin or mucous membranes caused by external contact with noxae that have an immunological, chemical or physical effect. The main triggers are allergens and irritant substances.

The peptides of the invention or the preparations in which they are present are therefore preferably used to prevent or reduce the penetration and/or accumulation of contact allergens on and/or in the skin.

The peptides form a skin barrier for the contact allergen. The peptides thus result in particular in the prevention or attenuation of allergic, in particular contact-allergic, reactions in or on the skin. Preference is thus also given in accordance with the invention to the prevention or attenuation of allergic contact dermatitis by the peptides of the invention or by the preparations in which they are present.

Preference is given to employing the peptides of the invention against chemical allergens in order to prevent especially allergies of type I and especially of type IV.

Chemical allergens include for example textile fibers, dyes, coating substances or other chemical substances.

Preference is given to employing the use and method according to the invention against these substance classes.

Particular preference is given to using the peptides of the invention against chemically electronegative allergens.

The peptides of the invention may be employed with preference against contact allergens.

Contact allergens that should preferably be prevented include inter alia metals such as nickel, preservatives, fragrances and dyes, rubber constituents, resin constituents, adhesives, monomers of plastics such as epoxy resin and acrylates, disinfectants, and particular natural substances such as plant substances in particular, especially thiomersal, perfume, cobalt, nickel, formaldehyde, urushiols, balsam of Peru, rosin, isothiazolinones, chromium or thiuram mix.

Preference is in accordance with the invention thus given to preventing or attenuating the development of a contact allergy and/or contact dermatitis on contact with allergens.

A further preferred embodiment of the invention is also the non-therapeutic, in particular cosmetic use of one or more peptides or of the preparation in which they are present as a skin barrier against foreign substances and/or allergens, especially contact allergens, and for skin care.

Another embodiment of the invention is a cosmetic method for protecting against allergens by applying to the skin a preparation comprising one or more peptides of the invention.

The non-therapeutic use and the cosmetic method of the peptides of the invention serve to prevent or reduce the penetration and/or accumulation of foreign substances and/or allergens, in particular contact allergens, on and/or in the skin. Thus, a contribution to skin care is inter alia also made.

The peptides of the invention are selected from the group of peptides having 2-10 amino acids and are characterized in that they contain one or more amino acids having one or more reactive side chains.

The peptides of the invention preferably contain at least one amino acid selected from the group histidine, cysteine, lysine, tyrosine, serine, selenocysteine, homocysteine, asparagine, aspartic acid, glutamine, glutamic acid, methionine, phenylalanine and/or pyrrolysine.

Preference is in turn given to selecting peptides from the group GSH, HHHHHH, Ac-RFAACAA, Ac-RFAALAA, RFAALAA, RFAACAA, Ac-RAACAA, RAACAA, Ac-RFACAA, RFACAA, Ac-RFACA and/or RFACA.

The sequence listing of the peptides that are preferred according to the invention is:

Seq. 1:
HHHHHH = His His His His His His
Seq. 2:
RFAACAA = Arg Phe Ala Ala Cys Ala Ala
Seq. 3:
RFAALAA = Arg Phe Ala Ala Leu Ala Ala
Seq. 4:
RAACAA = Arg Ala Ala Cys Ala Ala
Seq. 5:
RFACAA = Arg Phe Ala Cys Ala Ala
Seq. 6:
RFACA = Arg Phe Ala Cys Ala

and in each case the acetylated acids thereof (Ac-).

For topical application, the peptides of the invention are preferably incorporated in cosmetic or dermatological preparations in order to achieve user-friendly topical application of the allergen barrier.

The invention is therefore also a preparation for topical application, in particular a cosmetic preparation, comprising one or more peptides from the group of peptides having 2-10 amino acids, wherein at least one of these amino acids has one or more reactive side chains.

The content of peptides of the invention having 2 to 10 amino acids is advantageously within a range from 0.01% to 10% by weight, especially within a range from 0.1% to 5% by weight, more preferably within a range from 0.2% to 5% by weight, based on the total mass of the preparation.

A method for protecting against allergens is thus possible by applying to the skin a preparation comprising one or more peptides, wherein the peptides are selected from the group of peptides having 2-10 amino acids and at least one of these amino acids has one or more reactive side chains.

The terms “skin barrier” and “barrier effect” encompass in accordance with the invention the interception, blocking, inactivation, prevention and reduction of penetration or accumulation of foreign substances such as allergens, in particular contact allergens, on or in the skin and/or making this more difficult. The substances giving rise to an undesired reaction are in accordance with the invention collectively referred to in simple terms as foreign substances and/or allergens.

Attachment or binding of the allergen to the peptide prevents or at least reduces accumulation or penetration of the allergen on or in the skin.

A reduction in the accumulation of allergens on the skin takes place in respect both of the amount of allergen and the rate.

In other words, one or more peptides of the invention are used to intercept, block and/or inactivate allergens and/or to prevent, reduce, or hinder the penetration or accumulation of foreign substances or allergens on or in the skin, in particular human skin.

Peptides of the invention intercept the foreign substances, individual substances such as metals or metal ions, proteases or allergens. They thus prevent, reduce or hinder the ability of these substances to accumulate on the skin or penetrate into the skin and to result there in undesirable reactions on or in the skin or the body. The triggering of an immune response is consequently prevented. More particularly, prevention of the penetration of allergens means that an allergic reaction is attenuated or prevented altogether.

In particular, this prevents atopic dermatitis and/or allergic contact dermatitis.

The peptides of the invention are selected from the group of peptides having 2-10 amino acids. The peptides are preferably selected from the group of up to 8 amino acids, especially fewer than 8 amino acids.

Peptides having a high number of amino acids run the risk of being classed as a foreign substance when they come into contact with the skin, and possibly triggering a specific immune defense via MHC groups.

The abbreviation MHC stands for “major histocompatibility complex”, a group of very varied genes that play a role in the immune response of vertebrates. The genes provide the instructions for the construction of transporter molecules. Their role is to hold on to protein fragments (peptides) from viruses and bacteria and present them to specialized cells of the immune system: this allows them to recognize and fight the enemies.

The peptides of the invention are further characterized in that they contain one or more amino acids that in turn have one or more reactive side chains.

Reactive side chains are characterized in that they contain electron donors and/or acceptors. These properties make it possible for such side chains to be involved in the formation of covalent bonds, coordinate bonds, hydrogen bonds or van der Waals interactions between allergen/irritant/foreign substance and peptide.

The peptides of the invention advantageously contain at least one amino acid having a free thiol group. The free thiol group is a reactive side chain such as that found in for example the amino acid cysteine.

Thus, the peptide glutathione (GSH) or γ-L-glutamyl-L-cysteinylglycine are also for example preferred peptides having a reactive side chain. γ-L-Glutamyl-L-cysteinylglycine is a tripeptide having the sequence glutamic acid-cysteine-glycine (Glu-Cys-Gly).

In accordance with the invention, a side chain can also be referred to as a group and thus characterizes the reactivity and thus allergen binding of peptides containing it.

When peptides that initially contain no reactive group, for example no thiol group, are used according to the invention, they are converted, through addition of one or more amino acids having reactive groups, for example cysteine, into a peptide of the invention that has a reactive group. The designation of the peptides of the invention can in that case still include the original peptide designation, this being extended by the addition of the reactive amino acid(s).

Given the standard designation for peptides without a thiol group, for example oligopeptide-6, e.g. KDIFTRALN, adding the amino acid cysteine results in an extended peptide of the invention having the sequence CKDIFTRALN, or Cys-oligopeptide-6.

The peptides of the invention advantageously contain one or more particularly chemically active amino acids such as histidine, cysteine, lysine, tyrosine, serine, selenocysteine, homocysteine, asparagine, aspartic acid, glutamine, glutamic acid, methionine, phenylalanine or pyrrolysine.

It has been found that allergens show a high affinity for binding to these reactive amino acids. Surprisingly, these specific peptides are thus able to bind the penetrating substances and be used in accordance with the invention.

The binding takes place in a wide variety of ways, for example by intermolecular interactions, ionic bonds, hydrogen bonds, van der Waals bonds or covalent bonds. Overlapping of different binding modes can also occur.

What is key is purely the effect thereby achieved—the barrier effect—that results in inactivation of allergens, preventing them from penetrating into the skin or from accumulating on the skin.

The accumulation of foreign substances/allergens on the skin is thus according to the invention reduced or prevented. Even when the inventive preparation and peptides are present on the skin onto which the foreign substances are bound, this is not to be understood as meaning accumulation in and on the skin.

By exploiting the affinity of allergens for the reactive amino acid side chains or groups, the use according to the invention opens up the means of preventing or attenuating an allergic skin reaction on contact with allergens that has long been sought.

A novel antiallergic agent/novel antiallergic method has according to the invention thus been created that does not eliminate or at least alleviate the symptoms of an allergic pathology, but rather prevents an allergic reaction from developing in the first place.

Examples of reactive amino acids are cysteine and methionine. Both contain a sulfur atom. Cysteine is a polar amino acid and has a free thiol group. This property of cysteine is very important for the structure of proteins, since it is how polypeptide chains are linked to one another or else covalent linkages are created within a polypeptide chain that considerably stabilize the conformation of a protein. The term cystine is sometimes also used for two cysteines connected via disulfide bridges. In metalloproteins, it is often a cysteine residue that binds zinc, copper, or iron atoms.

Hexahistidine is also important in the binding of metalloproteins.

This mode of action is also utilized in the coupling of nickel, thus classic nickel allergy is attenuated or even prevented altogether by applying a peptide of the invention.

The peptides of the invention can advantageously contain both naturally occurring amino acids and also amino acids synthetically produced and modified, for example by acetylation.

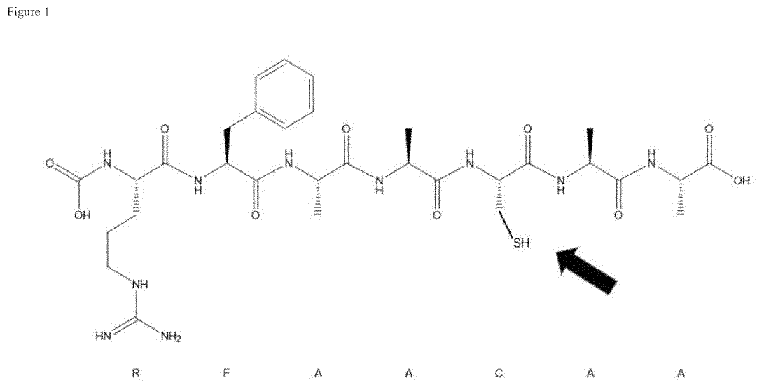

Examples of preferred peptides are
Glutathione (GSH), also known as 7-L-glutamyl-L-cysteinylglycine, is a tripeptide having the sequence glutamic acid-cysteine-glycine.
Hexahistidine (HHHHHH)
Ac-RFAACAA—a peptide having the sequence acetylarginine-phenylalanine-alanine-alanine-cysteine-alanine-alanine
The peptide is shown by way of example in FIG. 1, in which the active amino acid side chain is marked with an arrow.
Ac-RFAALAAA—a peptide having the sequence acetylarginine-phenylalanine-alanine-alanine-lysine-alanine-alanine
Ac-RAACAA
Ac-RFACAA
Ac-RFACA
RFAALAA
RFAACAA
RAACAA
RFACAA
RFACA

The peptide sequence listing of these peptides is given in the appendix.

For use, one or more inventive peptides that contain one or more active amino acids are applied to the surface of the skin.

The invention therefore also encompasses preparations for topical application, in particular cosmetic or dermatological preparations, comprising one or more of the peptides of the invention.

Topical application can take place in many different ways, for example in the form of a cosmetic formulation. Application to the skin results in the creation of a protective mechanism, a barrier effect.

Simple aqueous or lipid-containing formulations up to preparation forms with a complex make-up are possible, depending on the field of use.

The skin is in constant contact with foreign substances from the environment, or allergens. The protective mechanism means that foreign substances from the environment now come up against this barrier first. Potential allergens encounter the peptides contained in the applied formulation and are thus able to directly react with or be bound or at least hindered by the active amino acids of these peptides. The allergens are thus intercepted and are no longer able to cause a biological reaction in the body.

The allergen reaction with effector (skin's own peptide or protein) is prevented before an allergy to the allergen can develop.

Likewise, the allergens are intercepted and the triggering of the allergy by the specific allergen (elicitation) thus prevented.

The barrier effect of the peptides of the invention and their prevention or attenuation of allergic reactions has been impressively demonstrated in the investigations elucidated below.

One operating principle in the experiments carried out here is based primarily on the scavenging of allergens through the formation of covalent bonds to the reactive group, for example the amino acid cysteine, according to the mechanism of a nucleophilic addition, also termed SN1 and SN2.

The following investigations were carried out to demonstrate the barrier effect.

It has been shown that both biological modification by allergens in the skin and biological signaling cascades are prevented.

The investigations were carried out using Phenion FTS full skin models (Phenion, Henkel). These were maintained for 3 days at 37° C. and 7% CO₂ in deep-well plates having a 3 μM pore size in the cell culture inserts. The skin models were maintained in 1.5 ml of air-liquid interface medium without phenol red. The medium was changed daily. The test substances were dissolved in ethanol and incubated at room temperature for 24 h. After culturing for 24 h, 25 μl of the substance solution was dripped onto a sterile filter paper that had previously been laid on the skin model. The barrier peptide (0.5 mM AcRFAACAA) had previously been applied to the model as a solution in 100 mM phosphate buffer (pH 7.5). Alternatively, the barrier peptide was applied in a formulation having a content of 0.25% by weight.

After culturing for a further 24 h, the models were harvested and one half embedded in OCT mounting medium (VWR Q-Path Chemicals), the other half snap-frozen in liquid nitrogen. The supernatant of each model was collected and stored at −20° C.

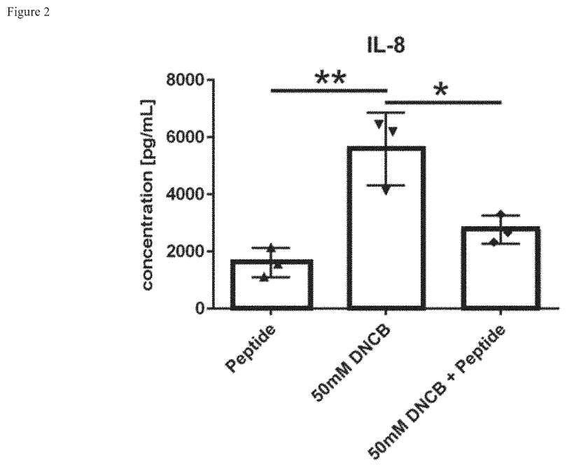

The supernatants were then analyzed (FIGS. 2, 3 and 4).

The supernatants of the Phenion FTS models were analyzed using the Bio-Plex Pro Human Cytokine Screening Panel (Bio-Rad) according to the manufacturer's protocol. This multiplex immunoassay is based on capture antibodies directed against the respective interleukins and covalently coupled to magnetic beads. The cytokine concentrations are calculated on the basis of the standard curves from the Bioplex system.

In FIGS. 2 to 4, the interleukin 8 (IL8) or interleukin 6 (IL6) concentration in pg/mL is plotted on the y-axis.

Both IL 8 and IL 6 are interleukins and thus messenger substances of the immune system. They are typically produced and released by skin cells when irritated and/or damaged by allergens and irritants.

The substances applied to the skin model are shown on the x-axis.

FIG. 2 shows a peptide of the invention (0.5 mM AcRFAACAA) on its own, 50 mM DNCB, and 50 mM DNCB plus peptide (0.5 mM AcRFAACAA).

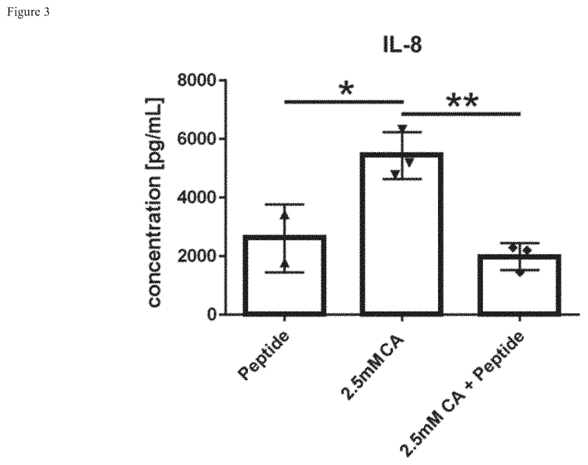

FIG. 3 shows the peptide on its own, 2.5 mM CA (cinnamaldehyde), and 2.5 mM cinnamaldehyde plus peptide.

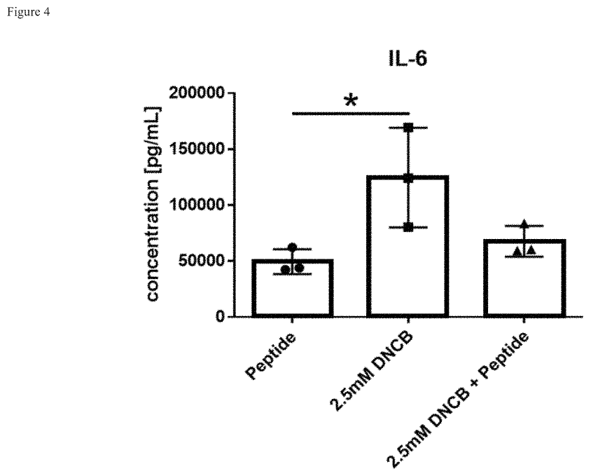

In FIG. 4, the interleukin 6 (IL6) concentration in pg/mL is plotted on the y-axis and the substances applied to the skin model (peptide on its own, 2.5 mM DNCB, and 2.5 mM DNCB plus peptide) are plotted on the x-axis.

DNCB (dinitrochlorobenzene) is a strong allergen. CA (cinnamaldehyde) is a moderate allergen. Eugenol is a phenylpropanoid with an intense cloves-like odor. Eugenol is both an antioxidant and a pro-oxidant. Its skin-irritant and allergy-promoting effect on skin and mucous membranes is based on the latter. All three allergens serve as model allergens for demonstrating the barrier effect and prevention or attenuation of allergic reactions of the peptides of the invention.

The unchanged skin model is clearly recognizable in FIGS. 2 to 4 (left bar in each case). By applying the allergens, a strong corrosive effect due to the allergens DNCB and CA (middle bar) is evident. Both IL 8 and IL 6 have been produced and released by the skin cells, which represents an allergic skin reaction. However, when the peptide was applied in combination with the allergen (right bar), the situation was surprisingly unchanged. The effect of the allergen was completely nullified by the peptide.

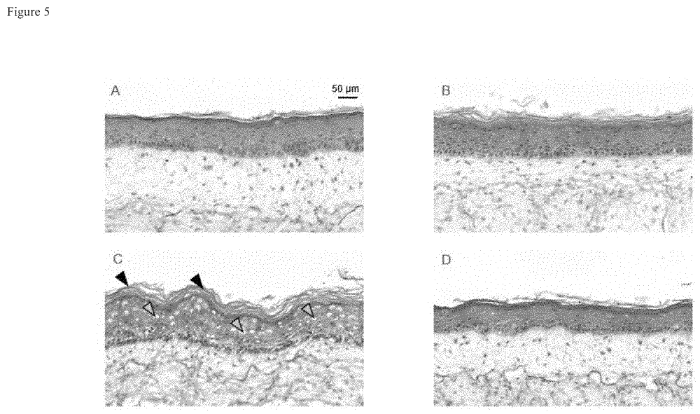

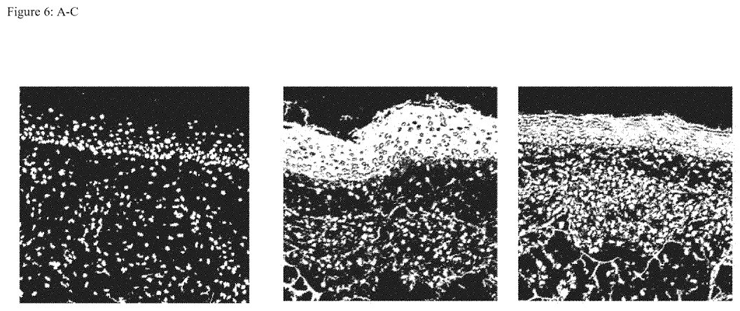

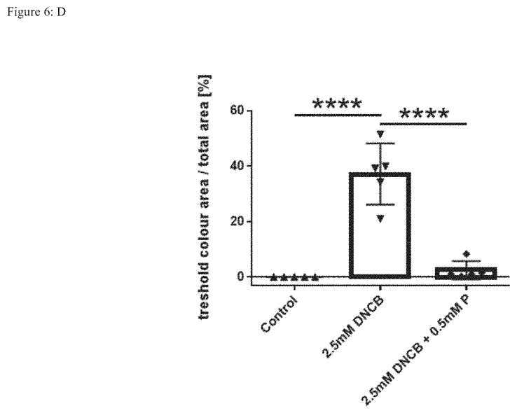

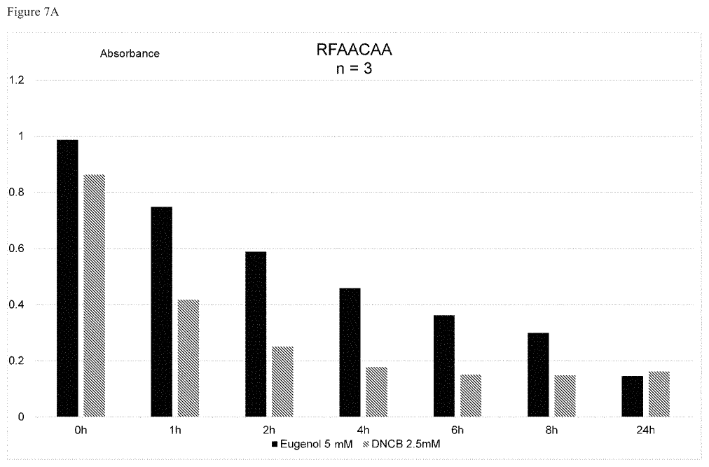

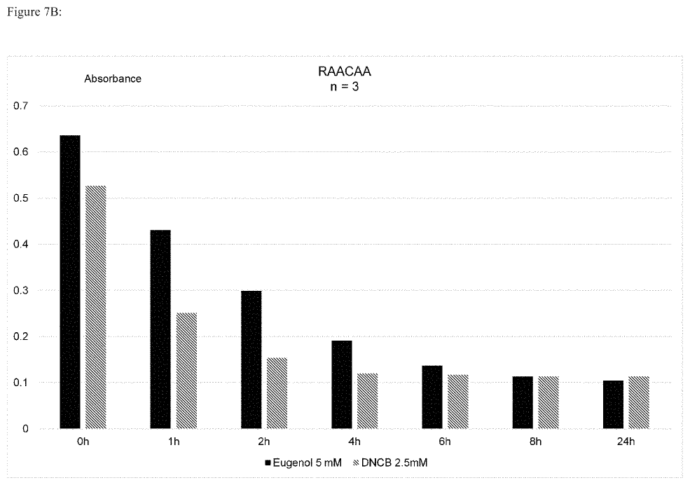

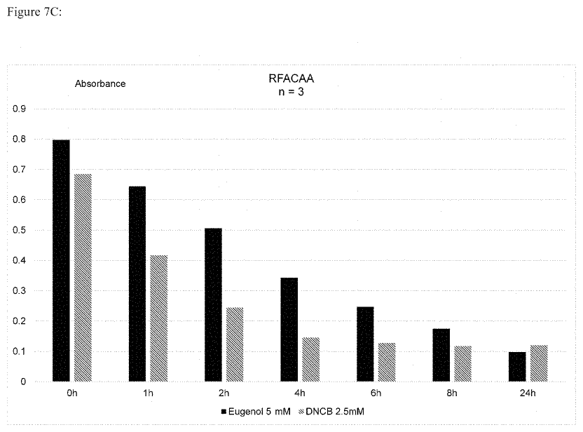

Further test results (immunohistochemistry) are shown in FIGS. 5 and 6.

For this, skin biopsies or skin models were embedded in OCT medium and frozen in the gas phase of liquid nitrogen. The frozen models were on the cryostat cut into sections with a layer thickness of 7 μm and applied to microscope slides. After air-drying for 1 to 24 h, they were fixed for 20 min in 3.7% paraformaldehyde at RT. After 3 PBS wash steps of 5 min each, the sections were blocked for 90 min at RT in a solution containing 0.2% Triton X-100. Primary antibodies (e.g. DNP, rabbit from Sigma (1:200)) were diluted appropriately and incubated with the sections for at least 4 h at RT. This was followed by three more PBS wash steps of 5 min each. The secondary antibody (1:800) and Hoechst Fluorescent Stain (1:2000) were incubated with the sections for 1-2 h at RT in the dark. After washing three more times, the sections were mounted with Fluoromount-G mounting medium (Sothern BioTech) and covered with a coverslip. Fluorescence microscopy was carried out using a Zeiss Axio Observer Z1.

The fluorescence images were analyzed with ImageJ. The DNP-stained areas were measured with the Threshold Color tool.

FIG. 5 shows the hematoxilin and eosin (H&E) staining of the cryosections, here in black and white, in the original in color.

FIG. 5 shows the H&E-stained cryosections of the Phenion skin models, A) after incubating with PBS (control), B) after incubating with 0.5 mM AcRFAACAA peptide (control), C) after incubating with 2.5 mM DNCB (strong allergen), and D) after incubating with DNCB and peptide.

The unchanged skin model in A) and B) and also a strong corrosive effect of DNCB in C) can be seen clearly. In addition to a dissolving stratum corneum, a strong loss of cell nuclei in the epidermis due to the effect of the allergen can be seen (arrows). In D) the situation is as in A) and B). The effect of the allergen was completely nullified by the peptide.

In further investigations, frozen sections were thawed and incubated for 10 min in 3.7% paraformaldehyde at RT. After three PBS wash steps and one minute in distilled water, they are stained for 3 min in hematoxylin solution. After washing with water and 0.1% HCl, the sections are stained for 3 min in eosin solution and dehydrated in an ethanol series of 70%, 95% and 100%. The sections were mounted and coverslipped in Leica CV Ultra mounting medium. The images were acquired using a Pannoramic Scan II (3DHistech).

FIG. 6 shows immunohistological analyses of cryosections from Phenion skin models,

A) after incubating with PBS (control),

B) after incubating with 2.5 mM DNCB (strong allergen), and

C) after incubating with DNCB and peptide (0.5 mM AcRFAACAA peptide).

The cell nucleus staining with DAPI (4,6-diamine-2-phenylindole) in blue (here gray dots) and proteins haptenized by DNCB in red (here white area).

FIG. 6 D shows the quantification of the red intensity of A, B, and C. The y-axis shows the red area above a threshold value in relation to the total area under consideration, with the substances applied to the skin model (peptide on its own, 2.5 mM DNCB, and 2.5 mM DNCB plus peptide) shown on the x-axis.

What can be seen clearly is the absence of red staining in A) (no allergen used), and in B) an intense red staining both of the epidermis and of parts of the dermis, which indicates a very strong reaction of DNCB with the proteins of the skin and thus strong haptenization. In C) extremely reduced red staining can be seen, which shows that the effect of the allergen was almost completely nullified by the use of the peptide. This is quantified in D). (Red, or white in black/white copy).

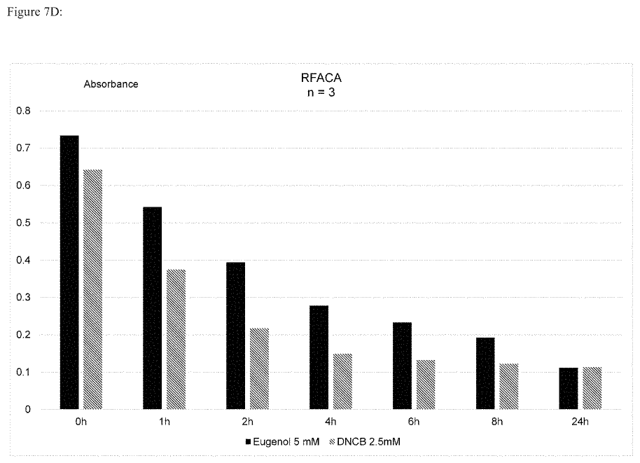

FIGS. 7 A-E, 8, and 9 show investigations into the reactivity of peptides of the invention with allergens over time (x-axis) and after 24 h. The absorbance is plotted on the y-axis. The peptide-allergen reactions were realized according to a direct peptide reactivity assay. The test is referred to as the in-chemico method, that is to say no cells are used, but a chemical reaction is carried out.

The direct peptide reaction assay (DPRA) is an OECD-validated test method.

The native, still-unbound peptide is detected by reaction with Ellman's reagent and corresponding absorbance measurement. The direct peptide reactivity assay (DPRA) is a chemical method for predicting epidermal protein binding. DPRA uses for example HPLC to measure the depletion of peptides in solution after exposure to test chemicals.

In the investigations carried out (FIG. 7), DPRA examines the reactivity of the test chemicals eugenol and DNCB with the peptides of the invention (in each case at 0.5 mM) and incubation for up to 24 hours. The measurement employed in the test is the rate of depletion of these peptides and is evaluated by means of a widely used HPLC-UV method.

RFAACAA, RAACAA, RFACAA, RFACA, and GSH were selected as exemplary representatives of the peptides of the invention, which represent the inventive use over the entire range of the peptides of the invention.

The reductions in absorbance due to the reaction of the peptide with allergen, eugenol (black), and DNCB (dashed) can be clearly seen in FIGS. 7 A to E, which demonstrates the inventive allergen-scavenging function of the peptides of the invention.

In the DPRA method, the absorbance at 415 nm is determined as the measured variable.

The allergen concentration is in the investigations carried out a standard 5 mM and thus 10 times the concentration of the peptide used (0.5 mM).

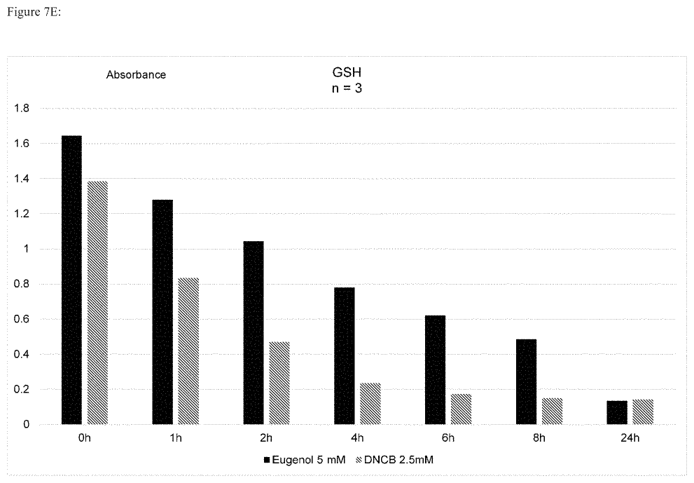

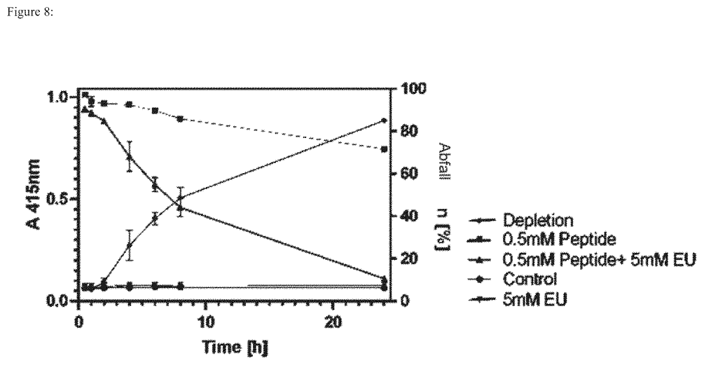

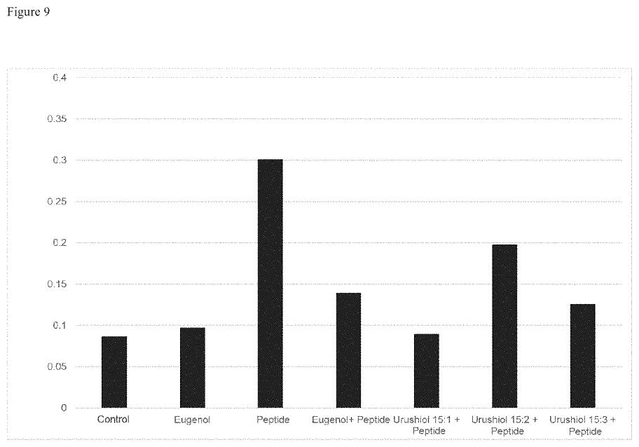

The reaction courses shown in FIGS. 8 and 9 were determined colorimetrically through the reaction of the peptide with Ellmann's reagent in a photometer. In this reaction, DTNB ((5,5′-dithiobis-2-nitrobenzoic acid, Ellmann's reagent) is cleaved and attached to the SH residue of the amino acid cysteine of the peptides of the invention (Ac-RFAACAA, RFAACAA).

The SH radical is at the same time the reactive binding site of the peptide with the allergens. The color reaction with DTNB (color change from colorless to yellow) competes with the allergen reaction. This means that the better the allergen reacts with the peptide, the less peptide remains available for the reaction with the detection reagent, i.e. the less the resulting yellow coloration. This measurement at 415 nm is shown in FIG. 9.

Alternatively, the decrease in the peptide in the allergen reaction can also be determined using a UV detector after HPLC separation, as shown in FIG. 8.

FIG. 8 shows the decrease (depletion in %) of the allergen eugenol (5 mM Eu) in the presence of the peptide Ac-RFAACAA (0.5 mM) over time (24 h) compared to the peptide on its own.

FIG. 9 shows the allergen reduction (eugenol, urushiols) in the direct peptide reaction assay (DPRA) after a 24 h reaction, n=3.

FIG. 9 shows the fall in peptide against eugenol and against various urushiol derivatives.

The control is the peptide in the buffer system without allergen, i.e. instead of allergen only its solvent (ethanol) is added.

Urushiols of the structure below were used.

| | | |
|---|---|---|
| Urushiol I Urushiol II Urushiol III Urushiol IV Urushiol V | 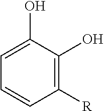 | —(CH₂)₁₄CH₃ —(CH₂)₇CH═CH(CH₂)₅CH₃ —(CH₂)₇CH═CHCH₂CH═CH(CH₂)₂CH₃ —(CH₂)₇CH═CHCH₂CH═CHCH═CHCH₃ —(CH₂)₇CH═CHCH₂CH═CHCH₂CH═CH₂ |

The investigated urushiols differ in the saturation in the side chain R.

Urushiols where R═C15 singly (C15:1), doubly (C15:2) or triply (C15:3) unsaturated were investigated.

The results demonstrate very high reactivity and thus protective activity of the peptides of the invention against urushiols too. The peptides, especially Ac-RFAACAA and RFAACAA, show very high reactivity and thus protective activity against urushiol structures I-V in particular.

Urushiols are the strongest naturally occurring allergen, which is in addition responsible for many sensitizations, as explained above. Urushiols are the main sensitizing constituent of the sumac plants, such as poison sumac (Toxicodendron quercifolium), including, in particular, in the various Rhus species such as poison ivy (Toxicodendron radicans), poison oak (Toxicodendron diversilobum), and lacquer tree (Rhus verniciflua), and also in Toxicodendron rydbergii, Toxicodendron toxicarium, and Toxicodendron vemix.

Allergy to urushiols, which requires prior sensitization, can be triggered in approx. 50-75% of all Americans. This contact dermatitis affects 10-50 million Americans every year.

Contact allergy to urushiols is a serious occupational disease, for example in agricultural and forestry workers and firefighters, but also in all recreation seekers who move around in nature. In the USA they are responsible for 7.1 million doctor visits and 430 000 hospital inpatient stays. For example, this contact allergy is the cause of 10% of all work time lost to injury at US Forest Services, and approximately one-third of forestry workers in California, Oregon, and Washington are unfit for work during the wildfire season because of these reactions.

The invention now provides a possible remedy here.

The preparations of the invention comprising the short-chain peptides of the invention result in alleviation of the allergic skin reaction to constituents of poison ivy.

All investigations impressively demonstrate the allergen barrier effect of the peptides of the invention in preventing or reducing the penetration and/or accumulation of foreign substances and/or allergens on and/or in the skin.

The numerous investigations of the various peptides demonstrate by way of example the barrier effect and the prevention or attenuation of allergic reactions for all peptides of the invention having a total of 2 to 10 amino acids, wherein at least one amino acid has one or more reactive side chains.

In addition to the pure demonstrations of efficacy, tests were also carried out with various concentrations of the allergens and peptides.

The allergens, such as for example DNCB, were tested in the range of 10; 5; 2.5; 0.5; 0.05; 0.005; 0.0005 mM, cinnamaldehyde in the range of 5; 2.5; 0.5; 0.05; 0.0005 mM, and eugenol in the range of 5, 2.5; 0.05; 0.0005 mM. The peptide concentrations were varied in the range of 4, 2, 0.5; 0.25 mM.

Significant barrier effects were achieved in all content ranges and ratios.

For instance, the peptide having the sequence HHHHHH=His His His His His His achieved a barrier effect against metals and against nickel in particular.

The peptides of the invention can advantageously be incorporated into typical cosmetic and dermatological preparations, which can take different forms. For instance, examples of preferred preparation forms are a solution, a water-in-oil (W/O) type emulsion or an oil-in-water (O/W) type emulsion, or multiple emulsions, for example a water-in-oil-in-water (W/O/W) type or oil-in-water-in-oil (O/W/O) type, a hydrodispersion or lipodispersion, a gel, a solid stick or even an aerosol.

The content of one or more peptides of the invention in preparations for topical application is advantageously within a range from 0.01% to 10% by weight, especially within a range from 0.1% to 5% by weight, more preferably within a range from 0.2% to 5% by weight, based on the total mass of the preparation.

Preferred application forms of the peptides of the invention are emulsion preparations, gels, and spray formulations.

Emulsions produced according to the invention, for example in the form of a cream, a lotion or a cosmetic milk, are advantageous and comprise, for example, fats, oils, waxes, and/or other fatty substances, and also water and one or more emulsifiers as are customarily used for formulations of this type.

Peptides are known to be susceptible to oxidation reactions and/or degradation reactions, which are made noticeable through discoloration or off-odors.

To improve the reactivity, i.e., the binding of allergens to the reactive groups of the peptides, it is advantageous to additionally add antioxidants and/or stabilizers to the preparations to protect the peptide.

The peptide-containing preparations of the invention advantageously comprise one or more antioxidants. The preparations of the invention therefore advantageously comprise one or more antioxidants selected from the group α-glycosylrutin, isoquercitin, tocopherol (vitamin E), citric acid, citrate buffer, BHT (butylated hydroxytoluene), and in particular EDTA (ethylenediaminetetraacetate).

The content of one or more antioxidants or stabilizers in the preparations for topical application is advantageously within a range from 0.01% to 2% by weight, especially within a range from 0.05% to 1.5% by weight, based on the total mass of the preparation.

The preparations of the invention also advantageously have a pH within a range from 4 to 8, especially within a range from 4 to 7.

A contribution to stabilization is surprisingly made by the pH of the preparation too. A preparation having a pH in the acidic range, advantageously of less than 7, especially of less than 6, has an advantageous effect on the stability of the peptide.

The pH can be adjusted using customary substances and buffer systems known to those skilled in the art.

The advantages of the addition of antioxidants and the choice of pH are thus firstly the improvement in stability and secondly a broader, more skin-friendly manner of use.

The preparations of the invention advantageously comprise one or more film formers.

In addition, the peptides can be chemically modified to improve the physicochemical properties. For example, derivatization with fatty acid residues (for example acetyl, caproyl, undecenoyl, or palmitoyl) increases the lipophilicity. Secondly, chemical modifications can help make the peptide more stable and thus protect it for example from degradation by peptidases.

So-called penetration enhancers, solubilizers and physical methods are able to increase the permeability of the skin and also further boost distribution in the skin.

An addition of solubilizers is therefore advantageous.

It is of course known to those skilled in the art that sophisticated cosmetic compositions are generally not conceivable without the customary auxiliaries and additives. These include for example consistency enhancers, fillers, dyes, emulsifiers, additional active substances such as vitamins or proteins, light stabilizers, stabilizers, insect repellents, alcohol, water, salts, EDTA, antimicrobial, proteolytic or keratolytic substances, etc., the addition of potential allergens being of course preferably avoided.

Mutatis mutandis, corresponding requirements apply to the formulation of medicinal preparations.

Allergy-triggering substances, allergens, are intercepted by the peptides of the invention consisting of 2-10 amino acids, wherein at least one amino acid has one or more reactive side chains. This barrier effect of the peptides, particularly in preparations for topical application, results in a novel form of antiallergic therapy.

### Example Preparations

The numerical values are contents by weight based on the total mass of the preparation.

| | 1 | 2 | 3 | 4 |
|---|---|---|---|---|
| INCI | m [%] | m [%] | m [%] | m [%] |
| Caprylic/Capric | 4.00 | 4.00 | 4.00 | 4.00 |
| Triglyceride | | | | |
| Cetyl Alcohol | 3.00 | 3.00 | 3.00 | 3.00 |
| Aqua + | 1.00 | 1.00 | 1.00 | 1.00 |
| Trisodium EDTA | | | | |
| BHT | 0.05 | 0.05 | 0.05 | 0.05 |
| Phenoxyethanol | 0.80 | 0.80 | 0.80 | 0.80 |
| Hydrogenated | 2.00 | 2.00 | 2.00 | 2.00 |
| Coco-Glycerides | | | | |
| Glycerol | 7.50 | 7.50 | 7.50 | 7.50 |
| Aqua + Sodium | 0.01 | 0.01 | 0.01 | 0.01 |
| Hydroxide | | | | |
| Alcohol Denat. + | 4.00 | 4.00 | 4.00 | 4.00 |
| Aqua | | | | |
| Xanthan Gum | 0.30 | 0.30 | 0.30 | 0.30 |
| Acrylates/C10-30 | 0.20 | 0.20 | 0.20 | 0.20 |
| Alkyl Acrylate | | | | |
| Crosspolymer | | | | |
| Glyceryl Stearate | 2.00 | 2.00 | 2.00 | 2.00 |
| Citrate | | | | |
| Dicaprylyl Ether | 4.00 | 4.00 | 4.00 | 4.00 |
| Aqua | to 100 | to 100 | to 100 | to 100 |
| Ac-RFAACAA | 0.25 | — | — | 0.2 |
| GSH | — | 1 | — | — |
| Ac-RFAALAA | — | — | 0.4 | 0.1 |

1.-21. (canceled)

22. A peptide, wherein the peptide is effective in the treatment of atopic dermatitis and/or allergic contact dermatitis and has 2 to 10 amino acids, at least one of these amino acids comprising one or more reactive side chains.

23. The peptide of claim 22, wherein at least one of the amino acids comprises one or more free thiol groups.

24. The peptide of claim 22, wherein at least one of the amino acids is histidine, cysteine, lysine, tyrosine, serine, selenocysteine, homocysteine, asparagine, aspartic acid, glutamine, glutamic acid, methionine, phenylalanine, pyrrolysine.

25. The peptide of claim 22, wherein the peptide is selected from GSH, HHHHHH, Ac-RFAACAA, Ac-RFAALAA, RFAALAA, RFAACAA, Ac-RAACAA, RAACAA, Ac-RFACAA, RFACAA, Ac-RFACA, RFACA.

26. A preparation, wherein the preparation is topically applicable and comprises one or more peptides according to claim 22.

27. The preparation of claim 26, wherein at least one peptide comprises at least one amino acid having one or more free thiol groups.

28. The preparation of claim 26, wherein at least one peptide comprises least one of histidine, cysteine, lysine, tyrosine, serine, selenocysteine, asparagine, aspartic acid, glutamine, glutamic acid, methionine, phenylalanine, pyrrolysine.

29. The preparation of claim 26, wherein at least one peptide is selected from GSH, HHHHHH, Ac-RFAACAA, Ac-RFAALAA, RFAALAA, RFAACAA, Ac-RAACAA, RAACAA, Ac-RFACAA, RFACAA, Ac-RFACA, RFACA.

30. The preparation of claim 26, wherein the preparation comprises from 0.01% to 10% by weight of the one or more peptides, based on a total weight of the preparation.

31. The preparation of claim 26, wherein the preparation is present as at least one of an emulsion, a gel or a spray.

32. The preparation of claim 26, wherein the preparation further comprises one or more antioxidants and/or stabilizers.

33. The preparation of claim 32, wherein the antioxidant(s) or stabilizer(s) are selected from α-glycosylrutin, isoquercitin, tocopherol (vitamin E), citric acid, citrate buffer, BHT (butylated hydroxytoluene), EDTA (ethylenediaminetetraacetate).

34. The preparation of claim 26, wherein the preparation has a pH of less than 7.

35. The preparation of claim 34, wherein the preparation has a pH of less than 6.

36. The preparation of claim 26, wherein the preparation is capable of preventing or reducing a penetration and/or accumulation of foreign substances and/or allergens on skin and/or in skin.

37. The preparation of claim 36, wherein the preparation is capable of preventing or reducing a penetration and/or accumulation of contact allergens on the skin and/or in the skin.

38. The preparation of claim 26, wherein the preparation is capable of preventing or reducing allergic reactions in or on the skin.

39. A method of forming barrier against foreign substances and/or allergens on skin, wherein the method comprises applying onto the skin the preparation of claim 26.

40. A method of preventing or reducing a penetration and/or accumulation of foreign substances and/or allergens on the skin and/or in the skin, wherein the method comprises applying onto the skin the preparation of claim 26.

41. A method of preventing or attenuating atopic dermatitis and/or allergic contact dermatitis, wherein the method comprises applying onto skin he preparation of claim 26.


SEQUENCE LISTING (6 sequences)

SEQ ID NO: 1 - protein, 6 aa, Artificial Sequence
  Peptide
HHHHHH

SEQ ID NO: 2 - protein, 7 aa, Artificial Sequence
  Peptide
RFAACAA

SEQ ID NO: 3 - protein, 7 aa, Artificial Sequence
  Peptide
RFAALAA

SEQ ID NO: 4 - protein, 6 aa, Artificial Sequence
  Peptide
RAACAA

SEQ ID NO: 5 - protein, 6 aa, Artificial Sequence
  Peptide
RFACAA

SEQ ID NO: 6 - protein, 5 aa, Artificial Sequence
  Peptide
RFACA